(19) United States
(12) Patent Application Publication
(10) Pub. No.: US 2026/0053382 A1
(43) Pub. Date: Feb. 26, 2026

(54) PHOTOPLETHYSMOGRAPHY BASED PHYSIOLOGICAL PARAMETER DETERMINATION TECHNIQUES USING EXISTING HEAD MOUNTED DEVICE HARDWARE

(71) Applicant: Meta Platforms Technologies, LLC, Menlo Park, CA (US)

(72) Inventors: Mantas Zurauskas, Redmond, WA (US); Christopher Bernard, San Jose, CA (US); Robert Dale Cavin, Bellevue, WA (US)

(21) Appl. No.: 19/306,572

(22) Filed: Aug. 21, 2025

Related U.S. Application Data
(60) Provisional application No. 63/686,474, filed on Aug. 23, 2024.

(51) Int. Cl. A61B 5/024; A61B 5/00; A61B 5/0205; A61B 5/021

(52) U.S. Cl. CPC A61B 5/02444; A61B 5/0205; A61B 5/021; A61B 5/6803; A61B 5/7271; A61B 2560/0462; A61B 2562/0219

(57) ABSTRACT

Methods, apparatuses, and systems for determining a heart rate of a user is provided. One such method comprises detecting, by a sensor communicatively coupled with the computing device, light reflecting from a region of interest of a face of a user during a time frame, determining by the computing device, based on the light that is detected, a sequence of light intensity values over the time frame, and determining, by the computing device, a heart rate of a user using the light intensity values.

### CROSS REFERENCE TO RELATED APPLICATION

This application claims the benefit of a U.S. Provisional application having U.S. Provisional Application No. 63/686,474, filed Aug. 23, 2024, the disclosure of which are incorporated by reference herein, in its entirety.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a flow diagram of an exemplary computer-implemented method for determining at least a physiological parameter specific to the user, according to some aspects of this disclosure;

FIG. 2 is an illustration of an example artificial-reality system according to some aspects of this disclosure;

FIG. 3 is an illustration of an example artificial-reality system with a handheld device according to some aspects of this disclosure;

FIG. 4A is an illustration of example user interactions within an artificial-reality system according to some aspects of this disclosure;

FIG. 4B is an illustration of example user interactions within an artificial-reality system according to some aspects of this disclosure;

FIG. 5 is an illustration of an example augmented-reality system according to some aspects of this disclosure;

FIG. 6A is an illustration of an example virtual-reality system according to some aspects of this disclosure;

FIG. 6B is an illustration of another perspective of the virtual-reality systems shown in FIG. 6A;

FIG. 7 is a block diagram showing system components of example artificial- and virtual-reality systems;

FIG. 8 is an illustration of an example system that incorporates an eye-tracking subsystem capable of tracking a user's eyes;

FIG. 9 is a more detailed illustration of various aspects of the eye-tracking subsystem illustrated in FIG. 8;

FIG. 10 depicts an example aspect of a physiological parameter determination system, some aspects of this disclosure;

FIG. 11 illustrates a filtered graphical representation, according to some aspects some aspects of this disclosure;

FIG. 12 depicts a frequency graphical representation derived from the filtered graphical representation illustrated in FIG. 11, according to some aspects some aspects of this disclosure; and

FIG. 13 depicts a method for determining the physiological parameter of the blood pressure of a user, according to some aspects of this disclosure.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

Head-mounted devices (HMDs) are widely used across a number of diverse fields such as gaming, entertainment, manufacturing, education, military, aviation, and healthcare, as well as a number of artificial intelligence (AI) based applications such as approximately real-time natural language translation, intelligent virtual assistants, adaptive user interfaces, emotion recognition, personalized training and simulation, automated quality control in industrial settings, and AI-driven augmented reality experiences for collaborative work and data visualization. In healthcare applications, HMDs are particularly useful for determining physiological data such as blood pressure, heart rate, and pulse rate. Obtaining and analyzing the underlying data for determining various physiological parameters, however, may involve the integration of additional hardware components, which can increase the size, weight, and power consumption of the device, ultimately reducing user comfort and system efficiency. Moreover, adding extra hardware complicates the software architecture, as the device must manage multiple component-specific applications and data streams, thereby overburdening memory and processing resources. The need for direct skin contact or precise sensor placement with conventional monitoring hardware further detracts from the user experience. While imaging photoplethysmography (iPPG) presents a promising non-contact approach for physiological monitoring, these techniques often suffer from inaccuracy caused by, e.g., variations in environmental lighting, susceptibility to movement artifacts, and the difficulty of consistently selecting and tracking a desirable region of interest (ROI) on the user's skin.

The techniques described in this disclosure address and overcome the above described deficiencies. In particular, these techniques facilitate non-contact, real-time monitoring and determination of physiological parameters such as heartbeat and blood pressure, using existing hardware and software architectures of HMDs. For example, by leveraging the data gathered by HMD cameras that are utilized for eye or face tracking, the system reduces the need for additional contact-based sensors, preserving device comfort, form factor, and user experience. This seamless integration also reduces hardware complexity, minimizes power consumption, and avoids the systemic inefficiencies associated with managing multiple sensor types, data streams, and prioritizing competing tasks.

Additionally, the techniques describe herein are advantageous in that they can dynamically and adaptively select and track a region of interest on the user's face, namely one that provides a robust dataset (e.g., light intensity values) for determining physiological parameters. These techniques, when implemented, compensate for user movement, facial expressions, and anatomical differences, ensuring robust and reliable signal quality. Advanced signal processing techniques, such as rolling average subtraction and frequency analysis, are also employed, which effectively filter out data that is less relevant for the determination of physiological parameters, e.g., data associated noise from ambient light fluctuations, motion artifacts, and sensor drift. In this way, these techniques allow for accurate extraction of physiological signals in various real-world conditions.

Furthermore, the non-invasive and unobtrusive nature of the system enhances user comfort and compliance, as there are no adhesive patches, finger clips, or other contact-based sensors required. The software-based approach allows for continuous, real-time monitoring across various HMD platforms and use cases. Moreover, the use of existing hardware and the integration of computationally efficient software, e.g., software for implementing one or more signal processing techniques, (1) ensures computationally efficient use of the memory included in HMDs, (2) enables efficient scalability and implementation of these techniques across various technologies areas (e.g., via installation of software updates across hundreds or thousands of HMDs used in various fields, e.g., fitness, telemedicine, remote health monitoring, fitness, and so forth, approximately in real time (e.g., within a few seconds or fractions of a second)), and (3) and facilitates rapid adaptation of newly developed features related to physiological monitoring capabilities via deployment of these features through software updates that do not involve hardware modifications.

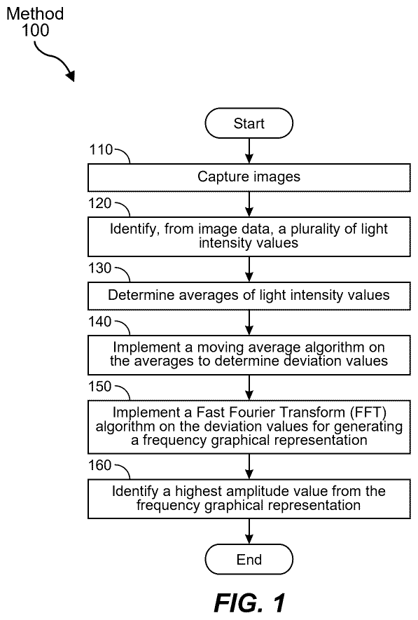

FIG. 1 is a flow diagram of a computer-implemented method 100 for determining at least a physiological parameter specific to the user, according to some aspects described and illustrated herein. The steps shown in FIG. 1 may be performed by any suitable computer-executable code and/or computing system, including the system(s) described later on in this disclosure and illustrated in FIGS. 1-4B. In one example, each of the steps shown in FIG. 1 may represent an algorithm whose structure includes and/or is represented by multiple sub-steps, examples of which will be provided in greater detail below.

As illustrated in FIG. 1, at step 110, one or more of the systems described herein, via one or more sensors operating as described in the present disclosure, captures images or a live video stream of the facial region of an individual. The system described herein may perform step 110 in a variety ways. For example, these systems utilize at least a camera positioned on a head mounted device (HMD) worn by a user to capture these images. In aspects, the camera is positioned such that the field of view of the camera includes, e.g., eyes, cheeks, temples, forehead, and the periocular region (skin surrounding the eyes) of the user.

At step 120, these systems described herein access image data associated with the image captured by the camera positioned of the HMD and identify a plurality of light intensity values. In particular, these systems identify light intensity values of pixels defining an area—a region of interest—as at least one of, e.g., the periocular region, cheeks, temples, forehead, and/or eyes. In aspects, these systems identify the region of interest as the area on the forehead directly above the eyes of the user. These systems may then identify the light intensity values associated with pixels that define this forehead area in each of the images (or each frame of the live video stream) captured by the camera.

At step 130, these systems determine an average value of the light intensity values associated with the pixels that define the forehead area for each of the images and generate a graphical representation in which these average values are plotted relative to various time values. Stated differently, the x-axis of the graphical representation includes a range of time values and the y-axis includes a range of byte values representative of light intensity. The byte values can range from 0 to 255 and correspond to grayscale values (if the camera that is used is a monochromatic camera) or color channel values of red, green, and blue if the camera used is a color camera.

At step 140, these systems implement a moving average or rolling average algorithm on the averages of the light intensity values determined at step 130 and determine a number of deviation values specific to each of the plurality of images that were captured. The systems will include these deviation values as part of an additional graphical representation (e.g., a filtered graphical representation), namely plotting the deviation values over a time range associated with the captured images.

At stage 150, these systems implement a Fast Fourier Transform (FFT) algorithm on the plurality of deviations determined in step 140, which results in a representation the deviations in a frequency domain. These systems generate a frequency graphical representation using the output of the FFT such that, frequency values derived from the time values plotted along the x-axis of the graphical representation from step 140 are plotted along the x-axis of the frequency graphical representation and amplitude values derived from the deviation values plotted along the y-axis of the filtered graphical representation are plotting along the y-axis of the frequency graphical representation. In aspects, in addition to the implementation of the FFT algorithm, these systems may utilize one or more artificial intelligence (AI) based machine learning (ML) models, implemented in conjunction with the FFT algorithm, to determine various physiological parameters specific to a user, e.g., heart rate, blood pressure, pulse rate, and so forth. For example, these systems may analyze a plurality of images or a live video stream of a region of interest, e.g., forehead region, and implement, approximately simultaneously, sequentially, or in accordance with a defined order, the FFT algorithm and/or one or more of a number of AI based ML models to predict physiological parameters specific to the user. In aspects, these models may utilize data specific to the user (e.g., inputs) that is gathered from, e.g., an accelerometer, a gyroscope, an inertial measurement unit (IMU), and so forth. Such components may be built into a HMD worn by the user. Some examples of the AI based ML models that may be utilized can include a convolutional neural network (CNN) or Long Short-Term Memory (LSTM). These models predict physiological parameters specific to the user by analyzing data from images and/or a live video stream in combination with data obtained from, e.g., e.g., an accelerometer, a gyroscope, an inertial measurement unit (IMU), and so forth.

Convolutional neural networks process input data having a spatial or grid-like structure, such as an image or multi-dimensional array. CNNs may include one or more convolutional layers, each applying a set of learned filters across the input to extract local features, such as edges, gradients, and textures. Subsequent layers may progressively combine such features to form higher-level representations corresponding to shapes, objects, or patterns. In aspects, CNNs may further comprise pooling layers for reducing dimensionality, normalization layers for stabilizing training, and fully connected layers for producing classification or regression outputs. The use of convolutional operations enables the network to learn spatial hierarchies of features without requiring manual feature engineering, thereby improving accuracy and efficiency in tasks including, but not limited to, image classification, object detection, or signal analysis.

Long short-term memory (LSTM) architectures may be utilized to perform various tasks, e.g., processing sequential or time-dependent data. The LSTM may include a plurality of memory cells, each configured with gating mechanisms including an input gate, a forget gate, and an output gate. These gates may selectively regulate the flow of information into, within, and out of the memory cell, thereby preserving relevant context while discarding less significant information. Such a structure allows the LSTM to maintain dependencies across both short and long temporal ranges, overcoming the vanishing gradient limitations associated with conventional recurrent neural networks. In aspects, the LSTM may be employed in applications such as natural language processing, speech recognition, predictive analytics, time-series forecasting, and a variety of other areas.

At stage 160, these systems identify at least an amplitude having a value that is higher than each of the remaining amplitude values and determines this value as corresponding to a physiological parameter specific to a user, e.g., a user's heartbeat.

Embodiments of the present disclosure may include or be implemented in conjunction with various types of Artificial Reality (AR) systems. AR may be any superimposed functionality and/or sensory-detectable content presented by an artificial-reality system within a user's physical surroundings. In other words, AR is a form of reality that has been adjusted in some manner before presentation to a user. AR can include and/or represent virtual reality (VR), augmented reality, mixed AR (MAR), or some combination and/or variation of these types of realities. Similarly, AR environments may include VR environments (including non-immersive, semi-immersive, and fully immersive VR environments), augmented-reality environments (including marker-based augmented-reality environments, markerless augmented-reality environments, location-based augmented-reality environments, and projection-based augmented-reality environments), hybrid-reality environments, and/or any other type or form of mixed- or alternative-reality environments.

AR content may include completely computer-generated content or computer-generated content combined with captured (e.g., real-world) content. Such AR content may include video, audio, haptic feedback, or some combination thereof, any of which may be presented in a single channel or in multiple channels (such as stereo video that produces a three-dimensional (3D) effect to the viewer). Additionally, in some embodiments, AR may also be associated with applications, products, accessories, services, or some combination thereof, that are used to, for example, create content in an artificial reality and/or are otherwise used in (e.g., to perform activities in) an artificial reality.

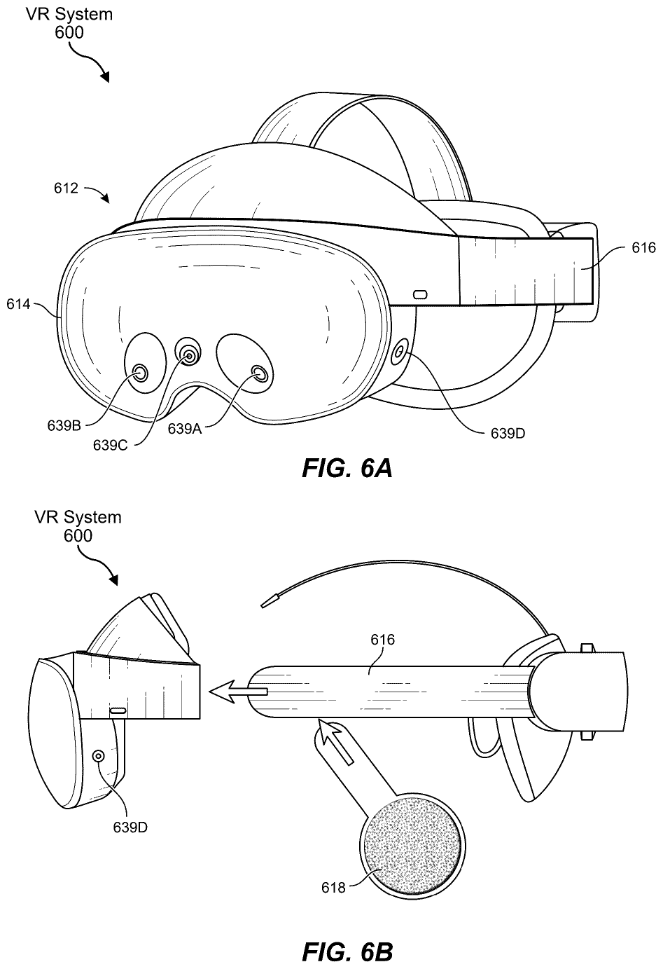

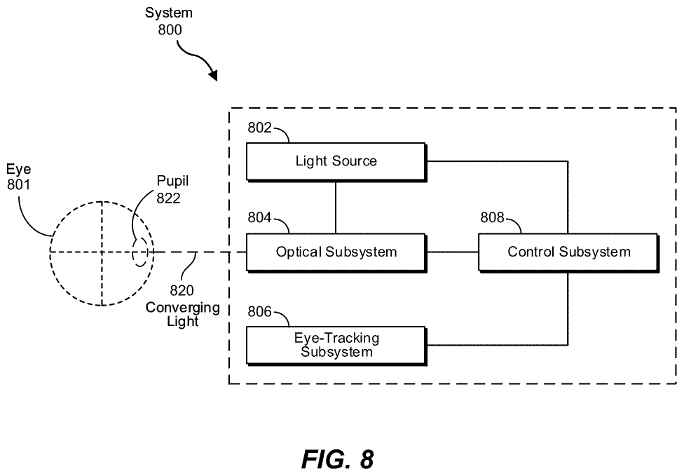

AR systems may be implemented in a variety of different form factors and configurations. Some AR systems may be designed to work without near-eye displays (NEDs). Other AR systems may include a NED that also provides visibility into the real world (such as, e.g., augmented-reality system 800 in FIG. 8) or that visually immerses a user in an artificial reality (such as, e.g., virtual-reality system 600 in FIGS. 6A and 6B). While some AR devices may be self-contained systems, other AR devices may communicate and/or coordinate with external devices to provide an AR experience to a user. Examples of such external devices include handheld controllers, mobile devices, desktop computers, devices worn by a user, devices worn by one or more other users, and/or any other suitable external system.

### Example Aspects

Aspect 1: A head-mounted device comprising a sensor that detects light emanating from a region of interest of a face of a user during a time frame, and a processor configured to determine, based on the light that is detected, a sequence of light intensity values over the time frame, and determines a heart rate of a user using the light intensity values.

Aspect 2: The head-mounted device of aspect 1, further comprising a camera, and the sensor that detects light being included as part of the camera.

Aspect 3: The head-mounted device of aspect 1 or aspect 2, wherein the processor determines the heart rate of the user using the light intensity values at least in part by determining average values of the light intensity values.

Aspect 4: The head-mounted device of any aspects 1-3, wherein the determining of the heart rate by the processor further comprises implementing, by the processor, a Fourier transform on the average values.

Aspect 5: The head-mounted device of any of aspects 1-4, wherein the determining of the heart rate by the processor further comprises the processor generating, based on the implementing of the Fourier transform on the average values, a plurality of frequencies and a plurality of amplitudes.

Aspect 6: The head-mounted device of any of aspects 1-5, wherein the processor is further configured to identify, as the heart rate of the user, an amplitude from the plurality of amplitudes that has a magnitude that is higher than respective magnitudes of a remaining amplitudes of the plurality of amplitudes.

Aspect 7: The head-mounted device of any of aspects 1-6, wherein the region of interest is associated with a forehead of the user.

Aspect 8: The head-mounted device of any of aspects 1-7, wherein the region of interest corresponds to at least one of the cheeks, temples, or periocular region of the user.

Aspect 9: A head-mounted device comprising a sensor that detects light emanating from a first region on a face of a user during a time frame, an additional sensor that detects additional light emanating from a second region of interest on the face of the user during the time frame, and a processor, wherein the processor determines light intensity values from the light that reflected from the first region, determines additional light intensity values from the additional light reflected from the additional region, and determines a heart rate of a user using the light intensity values or the additional light intensity values.

Aspect 10: The head-mounted device of aspect 9, wherein the determining of the heart rate by the processor comprises the processor comparing the light intensity values associated with the first region to the additional light intensity values associated with the additional region.

Aspect 11: The head-mounted device of aspect 9 or aspect 10, wherein the determining of the heart rate by the processor further comprises the processor discarding the additional light intensity values responsive to the additional light intensity values being lower than the light intensity values.

Aspect 12: The head-mounted device of any of aspects 9-11, wherein the determining of the heart rate by the processor further comprises the processor determining average values of the light intensity values, and implementing a Fourier transform on the average values.

Aspect 13: The head-mounted device of any of aspects 9-12, wherein the determining of the heart rate by the processor further comprises the processor generating, based on the implementing of the Fourier transform on the average values, a plurality of frequencies and a plurality of amplitudes, and identifying as the heart rate of the user, an amplitude from the plurality of amplitudes that has a magnitude that is higher than respective magnitudes of a remaining amplitudes of the plurality of amplitudes.

Aspect 14: The head-mounted device of any of aspects 9-13, wherein the first region and the second region are located on the face of the user.

Aspect 15: The head-mounted device of any of aspects 9-14, wherein the first region corresponds to at least one of the cheeks, temples, or periocular region of the user.

Aspect 16: A method implementing by a computing device, the method comprising detecting, by a camera communicatively coupled with the computing device, light reflecting from a region of interest of a face of a user during a time frame, determining by the computing device, based on the light that is detected, a sequence of light intensity values over the time frame, and determining, by the computing device, a heart rate of a user using the light intensity values.

Aspect 17: The method of aspect 16, wherein the determining of the heart rate comprises determining average values of the light intensity values, and implementing a Fourier transform on the average values.

Aspect 18: The method of aspect 16 or aspect 17, wherein the determining of the heart rate further comprises generating by the computing device, based on the implementing of the Fourier transform on the average values, a plurality of frequencies and a plurality of amplitudes.

Aspect 19: The method of any of aspects 16-18, further comprising identifying as the heart rate of the user, by the computing device, an amplitude from the plurality of amplitudes that has a magnitude that is higher than respective magnitudes of a remaining amplitudes of the plurality of amplitudes.

Aspect 20: The method of any of aspects 16-19, further comprising detecting, by an inertial measurement unit communicatively coupled to the computing device, inertial data specific to the user within a particular time frame, processing, by the computing device, the inertial data specific to the user and a plurality of images captured by the camera, the processing including implementing an artificial intelligence based machine learning model on the inertial data and the plurality of images, and generating responsive to the processing, by the computing device, at least a blood pressure value specific to the user.

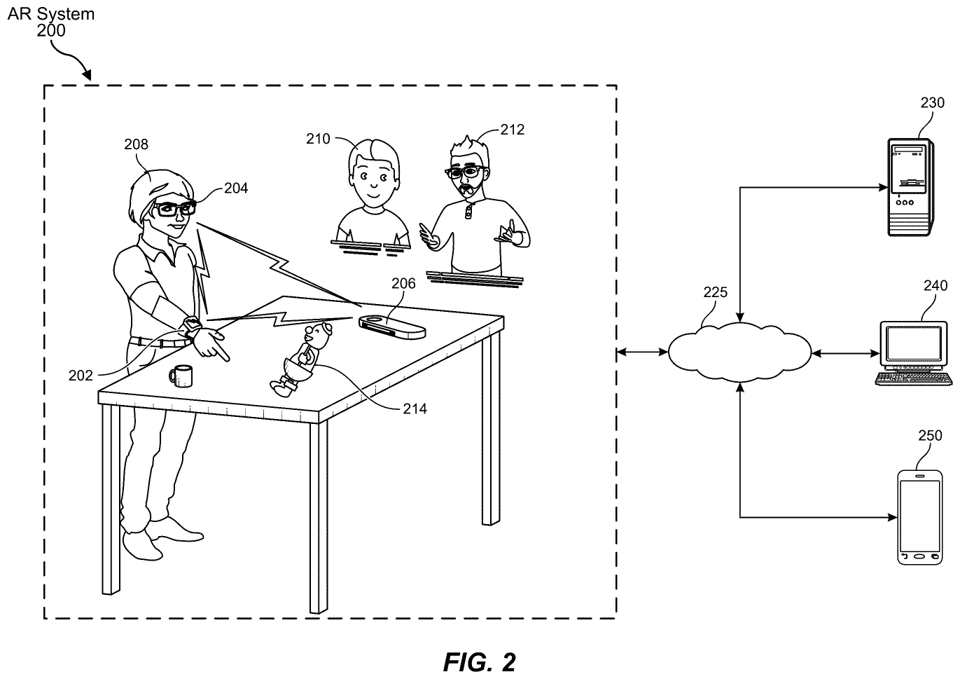

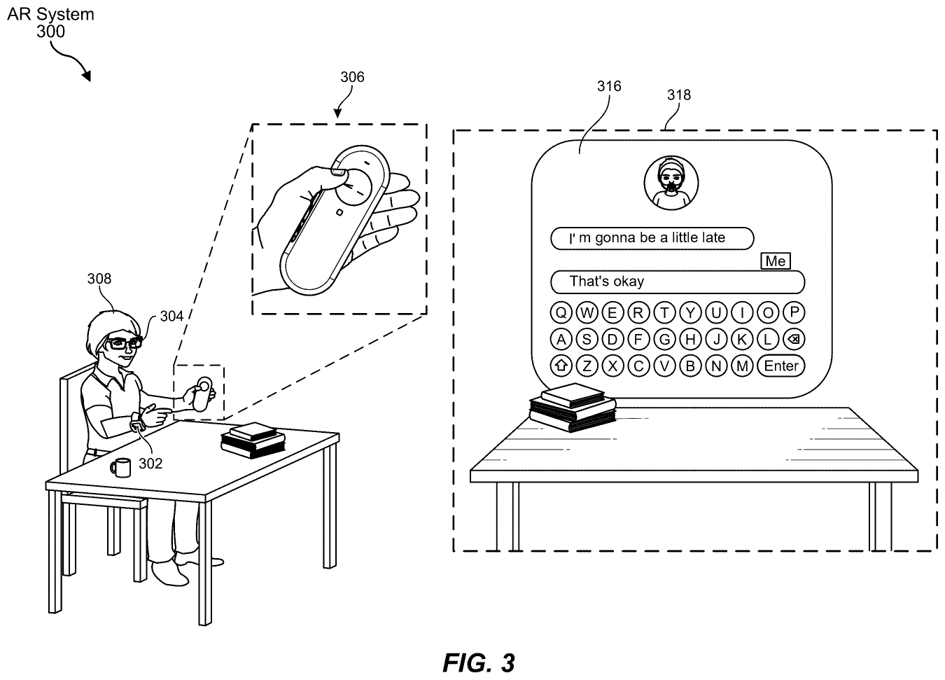

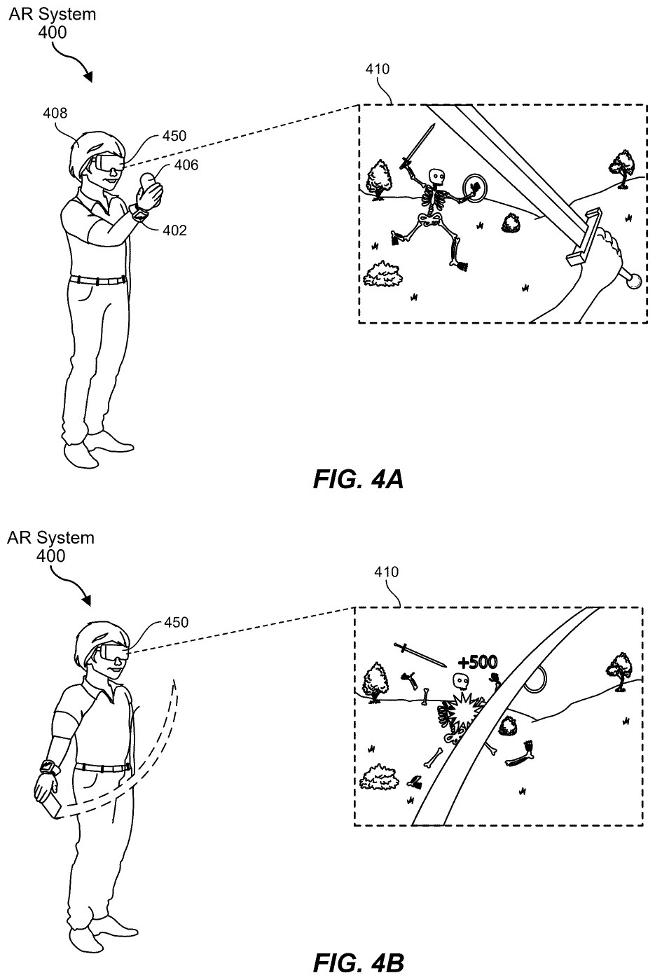

FIGS. 2-5 illustrate example artificial-reality (AR) systems in accordance with some embodiments. FIG. 2 shows a first AR system 200 and first example user interactions using a wrist-wearable device 202, a head-wearable device (e.g., AR glasses of AR System 800), and/or a handheld intermediary processing device (HIPD) 206. FIG. 3 shows a second AR system 300 and second example user interactions using a wrist-wearable device 302, AR glasses 304, and/or an HIPD 306. FIGS. 4A and 4B show a third AR system 400 and third example user 408 interactions using a wrist-wearable device 402, a head-wearable device (e.g., VR headset 450), and/or an HIPD 406.

Referring to FIG. 2, wrist-wearable device 202, AR glasses 204, and/or HIPD 206 can communicatively couple via a network 225 (e.g., cellular, near field, Wi-Fi, personal area network, wireless LAN, etc.). Additionally, wrist-wearable device 202, AR glasses 204, and/or HIPD 206 can also communicatively couple with one or more servers 230, computers 240 (e.g., laptops, computers, etc.), mobile devices 250 (e.g., smartphones, tablets, etc.), and/or other electronic devices via network 225 (e.g., cellular, near field, Wi-Fi, personal area network, wireless LAN, etc.).

In FIG. 2, a user 208 is shown wearing wrist-wearable device 202 and AR glasses 204 and having HIPD 206 on their desk. The wrist-wearable device 202, AR glasses 204, and HIPD 206 facilitate user interaction with an AR environment. In particular, as shown by first AR system 200, wrist-wearable device 202, AR glasses 204, and/or HIPD 206 cause presentation of one or more avatars 210, digital representations of contacts 212, and virtual objects 214. As discussed below, user 208 can interact with one or more avatars 210, digital representations of contacts 212, and virtual objects 214 via wrist-wearable device 202, AR glasses 204, and/or HIPD 206.

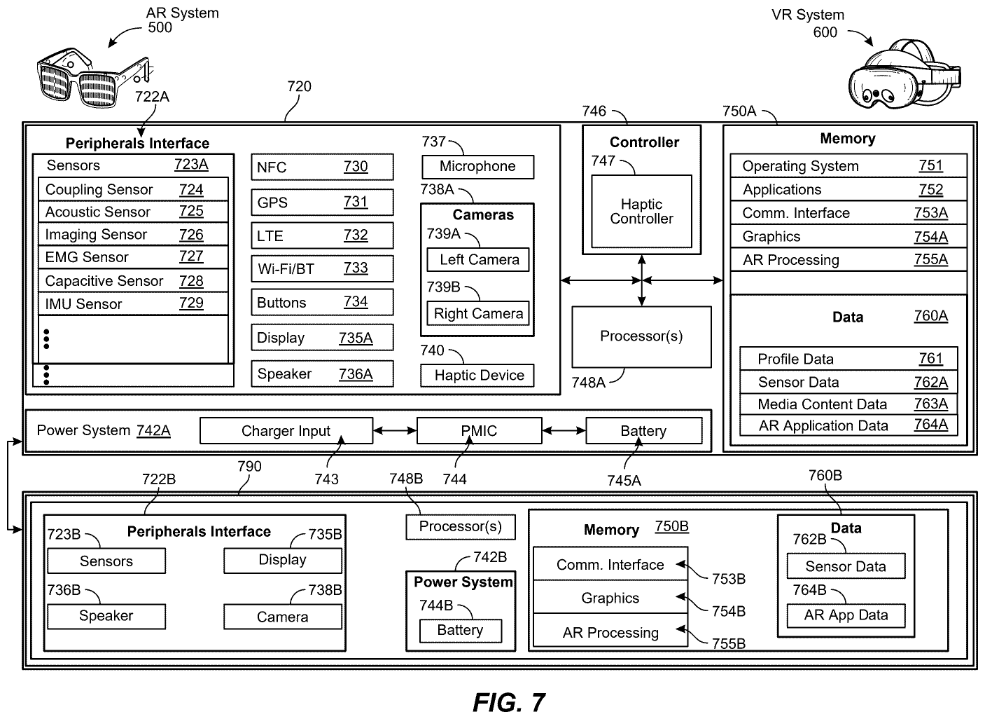

User 208 can use any of wrist-wearable device 202, AR glasses 204, and/or HIPD 206 to provide user inputs. For example, user 208 can perform one or more hand gestures that are detected by wrist-wearable device 202 (e.g., using one or more EMG sensors and/or IMUs, described below in reference to FIGS. 6 and 7) and/or AR glasses 204 (e.g., using one or more image sensor or camera, described below in reference to FIGS. 8-10) to provide a user input. Alternatively, or additionally, user 208 can provide a user input via one or more touch surfaces of wrist-wearable device 202, AR glasses 204, HIPD 206, and/or voice commands captured by a microphone of wrist-wearable device 202, AR glasses 204, and/or HIPD 206. In some embodiments, wrist-wearable device 202, AR glasses 204, and/or HIPD 206 include a digital assistant to help user 208 in providing a user input (e.g., completing a sequence of operations, suggesting different operations or commands, providing reminders, confirming a command, etc.). In some embodiments, user 208 can provide a user input via one or more facial gestures and/or facial expressions. For example, cameras of wrist-wearable device 202, AR glasses 204, and/or HIPD 206 can track eyes of user 208 for navigating a user interface.

Wrist-wearable device 202, AR glasses 204, and/or HIPD 206 can operate alone or in conjunction to allow user 208 to interact with the AR environment. In some embodiments, HIPD 206 is configured to operate as a central hub or control center for the wrist-wearable device 202, AR glasses 204, and/or another communicatively coupled device. For example, user 208 can provide an input to interact with the AR environment at any of wrist-wearable device 202, AR glasses 204, and/or HIPD 206, and HIPD 206 can identify one or more back-end and front-end tasks to cause the performance of the requested interaction and distribute instructions to cause the performance of the one or more back-end and front-end tasks at wrist-wearable device 202, AR glasses 204, and/or HIPD 206. In some embodiments, a back-end task is a background processing task that is not perceptible by the user (e.g., rendering content, decompression, compression, etc.), and a front-end task is a user-facing task that is perceptible to the user (e.g., presenting information to the user, providing feedback to the user, etc.). HIPD 206 can perform the back-end tasks and provide wrist-wearable device 202 and/or AR glasses 204 operational data corresponding to the performed back-end tasks such that wrist-wearable device 202 and/or AR glasses 204 can perform the front-end tasks. In this way, HIPD 206, which has more computational resources and greater thermal headroom than wrist-wearable device 202 and/or AR glasses 204, performs computationally intensive tasks and reduces the computer resource utilization and/or power usage of wrist-wearable device 202 and/or AR glasses 204.

In the example shown by first AR system 200, HIPD 206 identifies one or more back-end tasks and front-end tasks associated with a user request to initiate an AR video call with one or more other users (represented by avatar 210 and the digital representation of contact 212) and distributes instructions to cause the performance of the one or more back-end tasks and front-end tasks. In particular, HIPD 206 performs back-end tasks for processing and/or rendering image data (and other data) associated with the AR video call and provides operational data associated with the performed back-end tasks to AR glasses 204 such that the AR glasses 204 perform front-end tasks for presenting the AR video call (e.g., presenting avatar 210 and digital representation of contact 212).

In some embodiments, HIPD 206 can operate as a focal or anchor point for causing the presentation of information. This allows user 208 to be generally aware of where information is presented. For example, as shown in first AR system 200, avatar 210 and the digital representation of contact 212 are presented above HIPD 206. In particular, HIPD 206 and AR glasses 204 operate in conjunction to determine a location for presenting avatar 210 and the digital representation of contact 212. In some embodiments, information can be presented a predetermined distance from HIPD 206 (e.g., within 5 meters). For example, as shown in first AR system 200, virtual object 214 is presented on the desk some distance from HIPD 206. Similar to the above example, HIPD 206 and AR glasses 204 can operate in conjunction to determine a location for presenting virtual object 214. Alternatively, in some embodiments, presentation of information is not bound by HIPD 206. More specifically, avatar 210, digital representation of contact 212, and virtual object 214 do not have to be presented within a predetermined distance of HIPD 206.

User inputs provided at wrist-wearable device 202, AR glasses 204, and/or HIPD 206 are coordinated such that the user can use any device to initiate, continue, and/or complete an operation. For example, user 208 can provide a user input to AR glasses 204 to cause AR glasses 204 to present virtual object 214 and, while virtual object 214 is presented by AR glasses 204, user 208 can provide one or more hand gestures via wrist-wearable device 202 to interact and/or manipulate virtual object 214.

FIG. 3 shows a user 308 wearing a wrist-wearable device 302 and AR glasses 304, and holding an HIPD 306. In second AR system 300, the wrist-wearable device 302, AR glasses 304, and/or HIPD 306 are used to receive and/or provide one or more messages to a contact of user 308. In particular, wrist-wearable device 302, AR glasses 304, and/or HIPD 306 detect and coordinate one or more user inputs to initiate a messaging application and prepare a response to a received message via the messaging application.

In some embodiments, user 308 initiates, via a user input, an application on wrist-wearable device 302, AR glasses 304, and/or HIPD 306 that causes the application to initiate on at least one device. For example, in second AR system 300, user 308 performs a hand gesture associated with a command for initiating a messaging application (represented by messaging user interface 316), wrist-wearable device 302 detects the hand gesture and, based on a determination that user 308 is wearing AR glasses 304, causes AR glasses 304 to present a messaging user interface 316 of the messaging application. AR glasses 304 can present messaging user interface 316 to user 308 via its display (e.g., as shown by a field of view 318 of user 308). In some embodiments, the application is initiated and executed on the device (e.g., wrist-wearable device 302, AR glasses 304, and/or HIPD 306) that detects the user input to initiate the application, and the device provides another device operational data to cause the presentation of the messaging application. For example, wrist-wearable device 302 can detect the user input to initiate a messaging application, initiate and run the messaging application, and provide operational data to AR glasses 304 and/or HIPD 306 to cause presentation of the messaging application. Alternatively, the application can be initiated and executed at a device other than the device that detected the user input. For example, wrist-wearable device 302 can detect the hand gesture associated with initiating the messaging application and cause HIPD 306 to run the messaging application and coordinate the presentation of the messaging application.

Further, user 308 can provide a user input provided at wrist-wearable device 302, AR glasses 304, and/or HIPD 306 to continue and/or complete an operation initiated at another device. For example, after initiating the messaging application via wrist-wearable device 302 and while AR glasses 304 present messaging user interface 316, user 308 can provide an input at HIPD 306 to prepare a response (e.g., shown by the swipe gesture performed on HIPD 306). Gestures performed by user 308 on HIPD 306 can be provided and/or displayed on another device. For example, a swipe gestured performed on HIPD 306 is displayed on a virtual keyboard of messaging user interface 316 displayed by AR glasses 304.

In some embodiments, wrist-wearable device 302, AR glasses 304, HIPD 306, and/or any other communicatively coupled device can present one or more notifications to user 308. The notification can be an indication of a new message, an incoming call, an application update, a status update, etc. User 308 can select the notification via wrist-wearable device 302, AR glasses 304, and/or HIPD 306 and can cause presentation of an application or operation associated with the notification on at least one device. For example, user 308 can receive a notification that a message was received at wrist-wearable device 302, AR glasses 304, HIPD 306, and/or any other communicatively coupled device and can then provide a user input at wrist-wearable device 302, AR glasses 304, and/or HIPD 306 to review the notification, and the device detecting the user input can cause an application associated with the notification to be initiated and/or presented at wrist-wearable device 302, AR glasses 304, and/or HIPD 306.

While the above example describes coordinated inputs used to interact with a messaging application, user inputs can be coordinated to interact with any number of applications including, but not limited to, gaming applications, social media applications, camera applications, web-based applications, financial applications, etc. For example, AR glasses 304 can present to user 308 game application data, and HIPD 306 can be used as a controller to provide inputs to the game. Similarly, user 308 can use wrist-wearable device 302 to initiate a camera of AR glasses 304, and user 308 can use wrist-wearable device 302, AR glasses 304, and/or HIPD 306 to manipulate the image capture (e.g., zoom in or out, apply filters, etc.) and capture image data.

Users may interact with the devices disclosed herein in a variety of ways. For example, as shown in FIGS. 4A and 4B, a user 408 may interact with an AR system 400 by donning a VR headset 450 while holding HIPD 406 and wearing wrist-wearable device 402. In this example, AR system 400 may enable a user to interact with a game 410 by swiping their arm. One or more of VR headset 450, HIPD 406, and wrist-wearable device 402 may detect this gesture and, in response, may display a sword strike in game 410.

Having discussed example AR systems, devices for interacting with such AR systems and other computing systems more generally will now be discussed in greater detail. Some explanations of devices and components that can be included in some or all of the example devices discussed below are explained herein for ease of reference. Certain types of the components described below may be more suitable for a particular set of devices, and less suitable for a different set of devices. But subsequent reference to the components explained here should be considered to be encompassed by the descriptions provided.

In some embodiments discussed below, example devices and systems, including electronic devices and systems, will be addressed. Such example devices and systems are not intended to be limiting, and one of skill in the art will understand that alternative devices and systems to the example devices and systems described herein may be used to perform the operations and construct the systems and devices that are described herein.

An electronic device may be a device that uses electrical energy to perform a specific function. An electronic device can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device may be a device that sits between two other electronic devices and/or a subset of components of one or more electronic devices and facilitates communication, data processing, and/or data transfer between the respective electronic devices and/or electronic components.

An integrated circuit may be an electronic device made up of multiple interconnected electronic components such as transistors, resistors, and capacitors. These components may be etched onto a small piece of semiconductor material, such as silicon. Integrated circuits may include analog integrated circuits, digital integrated circuits, mixed signal integrated circuits, and/or any other suitable type or form of integrated circuit. Examples of integrated circuits include application-specific integrated circuits (ASICs), processing units, central processing units (CPUs), co-processors, and accelerators.

Analog integrated circuits, such as sensors, power management circuits, and operational amplifiers, may process continuous signals and perform analog functions such as amplification, active filtering, demodulation, and mixing. Examples of analog integrated circuits include linear integrated circuits and radio frequency circuits.

Digital integrated circuits, which may be referred to as logic integrated circuits, may include microprocessors, microcontrollers, memory chips, interfaces, power management circuits, programmable devices, and/or any other suitable type or form of integrated circuit. In some embodiments, examples of integrated circuits include central processing units (CPUs),

Processing units, such as CPUs, may be electronic components that are responsible for executing instructions and controlling the operation of an electronic device (e.g., a computer). There are various types of processors that may be used interchangeably, or may be specifically required, by embodiments described herein. For example, a processor may be: (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) an accelerator, such as a graphics processing unit (GPU), designed to accelerate the creation and rendering of images, videos, and animations (e.g., virtual-reality animations, such as three-dimensional modeling); (iv) a field-programmable gate array (FPGA) that can be programmed and reconfigured after manufacturing and/or can be customized to perform specific tasks, such as signal processing, cryptography, and machine learning; and/or (v) a digital signal processor (DSP) designed to perform mathematical operations on signals such as audio, video, and radio waves. One or more processors of one or more electronic devices may be used in various embodiments described herein.

Memory generally refers to electronic components in a computer or electronic device that store data and instructions for the processor to access and manipulate. Examples of memory can include: (i) random access memory (RAM) configured to store data and instructions temporarily; (ii) read-only memory (ROM) configured to store data and instructions permanently (e.g., one or more portions of system firmware, and/or boot loaders) and/or semi-permanently; (iii) flash memory, which can be configured to store data in electronic devices (e.g., USB drives, memory cards, and/or solid-state drives (SSDs)); and/or (iv) cache memory configured to temporarily store frequently accessed data and instructions. Memory, as described herein, can store structured data (e.g., SQL databases, MongoDB databases, GraphQL data, JSON data, etc.). Other examples of data stored in memory can include (i) profile data, including user account data, user settings, and/or other user data stored by the user, (ii) sensor data detected and/or otherwise obtained by one or more sensors, (iii) media content data including stored image data, audio data, documents, and the like, (iv) application data, which can include data collected and/or otherwise obtained and stored during use of an application, and/or any other types of data described herein.

Controllers may be electronic components that manage and coordinate the operation of other components within an electronic device (e.g., controlling inputs, processing data, and/or generating outputs). Examples of controllers can include: (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs.

A power system of an electronic device may be configured to convert incoming electrical power into a form that can be used to operate the device. A power system can include various components, such as (i) a power source, which can be an alternating current (AC) adapter or a direct current (DC) adapter power supply, (ii) a charger input, which can be configured to use a wired and/or wireless connection (which may be part of a peripheral interface, such as a USB, micro-USB interface, near-field magnetic coupling, magnetic inductive and magnetic resonance charging, and/or radio frequency (RF) charging), (iii) a power-management integrated circuit, configured to distribute power to various components of the device and to ensure that the device operates within safe limits (e.g., regulating voltage, controlling current flow, and/or managing heat dissipation), and/or (iv) a battery configured to store power to provide usable power to components of one or more electronic devices.

Peripheral interfaces may be electronic components (e.g., of electronic devices) that allow electronic devices to communicate with other devices or peripherals and can provide the ability to input and output data and signals. Examples of peripheral interfaces can include (i) universal serial bus (USB) and/or micro-USB interfaces configured for connecting devices to an electronic device, (ii) Bluetooth interfaces configured to allow devices to communicate with each other, including Bluetooth low energy (BLE), (iii) near field communication (NFC) interfaces configured to be short-range wireless interfaces for operations such as access control, (iv) POGO pins, which may be small, spring-loaded pins configured to provide a charging interface, (v) wireless charging interfaces, (vi) GPS interfaces, (vii) Wi-Fi interfaces for providing a connection between a device and a wireless network, and/or (viii) sensor interfaces.

Sensors may be electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device), (ii) biopotential-signal sensors, (iii) inertial measurement units (e.g., IMUs) for detecting, for example, angular rate, force, magnetic field, and/or changes in acceleration, (iv) heart rate sensors for measuring a user's heart rate, (v) SpO2 sensors for measuring blood oxygen saturation and/or other biometric data of a user, (vi) capacitive sensors for detecting changes in potential at a portion of a user's body (e.g., a sensor-skin interface), and/or (vii) light sensors (e.g., time-of-flight sensors, infrared light sensors, visible light sensors, etc.).

Biopotential-signal-sensing components may be devices used to measure electrical activity within the body (e.g., biopotential-signal sensors). Some types of biopotential-signal sensors include (i) electroencephalography (EEG) sensors configured to measure electrical activity in the brain to diagnose neurological disorders, (ii) electrocardiography (ECG or EKG) sensors configured to measure electrical activity of the heart to diagnose heart problems, (iii) electromyography (EMG) sensors configured to measure the electrical activity of muscles and to diagnose neuromuscular disorders, and (iv) electrooculography (EOG) sensors configured to measure the electrical activity of eye muscles to detect eye movement and diagnose eye disorders.

An application stored in memory of an electronic device (e.g., software) may include instructions stored in the memory. Examples of such applications include (i) games, (ii) word processors, (iii) messaging applications, (iv) media-streaming applications, (v) financial applications, (vi) calendars. (vii) clocks, and (viii) communication interface modules for enabling wired and/or wireless connections between different respective electronic devices (e.g., IEEE 802.15.4, Wi-Fi, ZigBee, 6LOWPAN, Thread, Z-Wave, Bluetooth Smart, ISA100.11a, WirelessHART, or MiWi), custom or standard wired protocols (e.g., Ethernet or HomePlug), and/or any other suitable communication protocols).

A communication interface may be a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, Bluetooth). In some embodiments, a communication interface can refer to a software layer that enables different software programs to communicate with each other (e.g., application programming interfaces (APIs), protocols like HTTP and TCP/IP, etc.).

A graphics module may be a component or software module that is designed to handle graphical operations and/or processes and can include a hardware module and/or a software module.

Non-transitory computer-readable storage media may be physical devices or storage media that can be used to store electronic data in a non-transitory form (e.g., such that the data is stored permanently until it is intentionally deleted or modified).

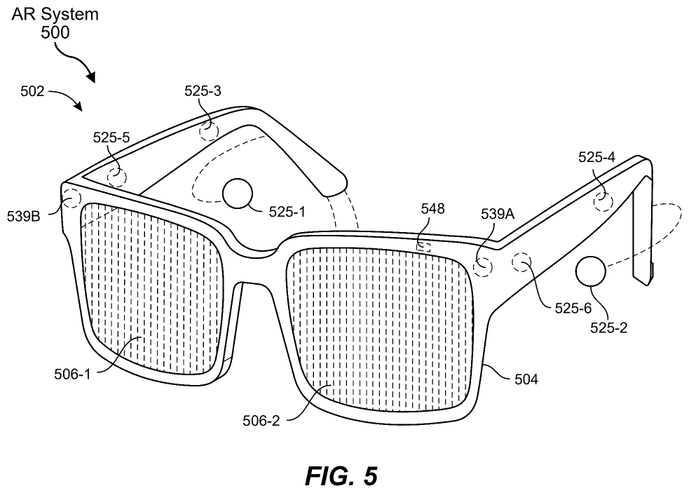

FIG. 5 is an illustration of an example augmented-reality system according to some embodiments of this disclosure. An example visual depiction of AR system 500, including an eyewear device 502 (which may also be described herein as augmented-reality glasses, and/or smart glasses). AR system 500 can include additional electronic components that are not shown in FIG. 5, such as a wearable accessory device and/or an intermediary processing device, in electronic communication or otherwise configured to be used in conjunction with the eyewear device 502. In some embodiments, the wearable accessory device and/or the intermediary processing device may be configured to couple with eyewear device 502 via a coupling mechanism in electronic communication with a coupling sensor 724 (FIG. 7), where coupling sensor 724 can detect when an electronic device becomes physically or electronically coupled with eyewear device 502. In some embodiments, eyewear device 502 can be configured to couple to a housing 790 (FIG. 7), which may include one or more additional coupling mechanisms configured to couple with additional accessory devices. The components shown in FIG. 5 can be implemented in hardware, software, firmware, or a combination thereof, including one or more signal-processing components and/or application-specific integrated circuits (ASICs).

Eyewear device 502 includes mechanical glasses components, including a frame 504 configured to hold one or more lenses (e.g., one or both lenses 506-1 and 506-2). One of ordinary skill in the art will appreciate that eyewear device 502 can include additional mechanical components, such as hinges configured to allow portions of frame 504 of eyewear device 502 to be folded and unfolded, a bridge configured to span the gap between lenses 506-1 and 506-2 and rest on the user's nose, nose pads configured to rest on the bridge of the nose and provide support for eyewear device 502, earpieces configured to rest on the user's ears and provide additional support for eyewear device 502, temple arms configured to extend from the hinges to the earpieces of eyewear device 502, and the like. One of ordinary skill in the art will further appreciate that some examples of AR system 500 can include none of the mechanical components described herein. For example, smart contact lenses configured to present artificial reality to users may not include any components of eyewear device 502.

Eyewear device 502 includes electronic components, many of which will be described in more detail below with respect to FIG. 7. Some example electronic components are illustrated in FIG. 8, including acoustic sensors 525-1, 525-2, 525-3, 525-4, 525-5, and 525-6, which can be distributed along a substantial portion of the frame 504 of eyewear device 502. Eyewear device 502 also includes a left camera 539A and a right camera 539B, which are located on different sides of the frame 504. Eyewear device 502 also includes a processor 548 (or any other suitable type or form of integrated circuit) that is embedded into a portion of the frame 504.

FIG. 6A is an illustration of an example virtual-reality system according to some embodiments of this disclosure and FIG. 6B is an illustration of another perspective of the virtual-reality systems shown in FIG. 6A.

FIGS. 6A and 6B show a VR system 600 that includes a head-mounted display (HMD) 612 (e.g., also referred to herein as an artificial-reality headset, a head-wearable device, a VR headset, etc.), in accordance with some embodiments. As noted, some artificial-reality systems (e.g., AR system 500) may, instead of blending an artificial reality with actual reality, substantially replace one or more of a user's visual and/or other sensory perceptions of the real world with a virtual experience (e.g., AR system 400).

HMD 612 includes a front body 614 and a frame 616 (e.g., a strap or band) shaped to fit around a user's head. In some embodiments, front body 614 and/or frame 616 include one or more electronic elements for facilitating presentation of and/or interactions with an AR and/or VR system (e.g., displays, IMUs and/or an accelerometer, gyroscope, tracking emitter or various types of other detectors). In some embodiments, HMD 612 includes output audio transducers (e.g., an audio transducer 618), as shown in FIG. 6B. In some embodiments, one or more components, such as the output audio transducer(s) 618 and frame 616, can be configured to attach and detach (e.g., are detachably attachable) to HMD 612 (e.g., a portion or all of frame 616, and/or audio transducer 618), as shown in FIG. 6B. In some embodiments, coupling a detachable component to HMD 612 causes the detachable component to come into electronic communication with HMD 612.

FIGS. 6A and 6B also show that VR system 600 includes one or more cameras, such as left camera 639A, right camera 639B, and centrally located camera 639C, which can be analogous to left and right cameras 639A and 639B on frame 504 of eyewear device 502. In some embodiments, VR system 600 includes one or more additional cameras (e.g., cameras 639C and 639D), which can be configured to augment image data obtained by left and right cameras 639A and 639B by providing more information. For example, camera 639C can be used to supply color information that is not discerned by cameras 639A and 639B. In some embodiments, one or more of cameras 639A to 639D can include an optional IR cut filter configured to remove IR light from being received at the respective camera sensors.

FIG. 7 illustrates a computing system 720 and an optional housing 790, each of which show components that can be included in AR system 500 and/or VR system 600. In some embodiments, more or fewer components can be included in optional housing 790 depending on practical restraints of the respective AR system being described.

In some embodiments, computing system 720 can include one or more peripherals interfaces 722A and/or optional housing 790 can include one or more peripherals interfaces 722B. Each of computing system 720 and optional housing 790 can also include one or more power systems 742A and 742B, one or more controllers 746 (including one or more haptic controllers 747), one or more processors 748A and 748B (as defined above, including any of the examples provided), and memory 750A and 750B, which can all be in electronic communication with each other. For example, the one or more processors 748A and 748B can be configured to execute instructions stored in memory 750A and 750B, which can cause a controller of one or more of controllers 746 to cause operations to be performed at one or more peripheral devices connected to peripherals interface 722A and/or 722B. In some embodiments, each operation described can be powered by electrical power provided by power system 742A and/or 742B.

In some embodiments, peripherals interface 722A can include one or more devices configured to be part of computing system 720, some of which have been defined above and/or described with respect to the wrist-wearable devices shown in FIGS. 6 and 7. For example, peripherals interface 722A can include one or more sensors 723A. Some example sensors 723A include one or more coupling sensors 724, one or more acoustic sensors 725, one or more imaging sensors 726, one or more EMG sensors 727, one or more capacitive sensors 728, one or more IMU sensors 729, and/or any other types of sensors explained above or described with respect to any other embodiments discussed herein.

In some embodiments, peripherals interfaces 722A and 722B can include one or more additional peripheral devices, including one or more NFC devices 730, one or more GPS devices 731, one or more LTE devices 732, one or more Wi-Fi and/or Bluetooth devices 733, one or more buttons 734 (e.g., including buttons that are slidable or otherwise adjustable), one or more speakers 736A and 736B, one or more microphones 737, one or more cameras 738A and 738B (e.g., including the left camera 739A and/or a right camera 739B), one or more haptic devices 740, and/or any other types of peripheral devices defined above or described with respect to any other embodiments discussed herein.

AR systems can include a variety of types of visual feedback mechanisms (e.g., presentation devices). For example, display devices in AR system 500 and/or VR system 500 can include one or more liquid-crystal displays (LCDs), light emitting diode (LED) displays, organic LED (OLED) displays, and/or any other suitable types of display screens. Artificial-reality systems can include a single display screen (e.g., configured to be seen by both eyes), and/or can provide separate display screens for each eye, which can allow for additional flexibility for varifocal adjustments and/or for correcting a refractive error associated with a user's vision. Some embodiments of AR systems also include optical subsystems having one or more lenses (e.g., conventional concave or convex lenses, Fresnel lenses, or adjustable liquid lenses) through which a user can view a display screen.

For example, respective displays 735A and 735B can be coupled to each of the lenses 506-1 and 506-2 of AR system 500. Displays 735A and 735B may be coupled to each of lenses 506-1 and 506-2, which can act together or independently to present an image or series of images to a user. In some embodiments, AR system 800 includes a single display 735A or 735B (e.g., a near-eye display) or more than two displays 735A and 735B. In some embodiments, a first set of one or more displays 735A and 735B can be used to present an augmented-reality environment, and a second set of one or more display devices 735A and 735B can be used to present a virtual-reality environment. In some embodiments, one or more waveguides are used in conjunction with presenting artificial-reality content to the user of AR system 800 (e.g., as a means of delivering light from one or more displays 735A and 735B to the user's eyes). In some embodiments, one or more waveguides are fully or partially integrated into the eyewear device 502. Additionally, or alternatively to display screens, some artificial-reality systems include one or more projection systems. For example, display devices in AR system 500 and/or VR system 600 can include micro-LED projectors that project light (e.g., using a waveguide) into display devices, such as clear combiner lenses that allow ambient light to pass through. The display devices can refract the projected light toward a user's pupil and can enable a user to simultaneously view both artificial-reality content and the real world. Artificial-reality systems can also be configured with any other suitable type or form of image projection system. In some embodiments, one or more waveguides are provided additionally or alternatively to the one or more display(s) 735A and 735B.

Computing system 720 and/or optional housing 790 of AR system 500 or VR system 600 can include some or all of the components of a power system 742A and 742B. Power systems 742A and 742B can include one or more charger inputs 743, one or more PMICs 744, and/or one or more batteries 745A and 744B.

Memory 750A and 750B may include instructions and data, some or all of which may be stored as non-transitory computer-readable storage media within the memories 750A and 750B. For example, memory 750A and 750B can include one or more operating systems 751, one or more applications 752, one or more communication interface applications 753A and 753B, one or more graphics applications 754A and 754B, one or more AR processing applications 755A and 755B, and/or any other types of data defined above or described with respect to any other embodiments discussed herein.

Memory 750A and 750B also include data 760A and 760B, which can be used in conjunction with one or more of the applications discussed above. Data 760A and 760B can include profile data 761, sensor data 762A and 762B, media content data 763A, AR application data 764A and 764B, and/or any other types of data defined above or described with respect to any other embodiments discussed herein.

In some embodiments, controller 746 of eyewear device 502 may process information generated by sensors 723A and/or 723B on eyewear device 502 and/or another electronic device within AR system 500. For example, controller 746 can process information from acoustic sensors 525-1 and 525-2. For each detected sound, controller 746 can perform a direction of arrival (DOA) estimation to estimate a direction from which the detected sound arrived at eyewear device 502 of AR system 500. As one or more of acoustic sensors 725 (e.g., the acoustic sensors 525-1, 525-2) detects sounds, controller 746 can populate an audio data set with the information (e.g., represented in FIG. 7 as sensor data 762A and 762B).

In some embodiments, a physical electronic connector can convey information between eyewear device 502 and another electronic device and/or between one or more processors 548, 748A, 748B of AR system 500 or VR system 600 and controller 746. The information can be in the form of optical data, electrical data, wireless data, or any other transmittable data form. Moving the processing of information generated by eyewear device 502 to an intermediary processing device can reduce weight and heat in the eyewear device, making it more comfortable and safer for a user. In some embodiments, an optional wearable accessory device (e.g., an electronic neckband) is coupled to eyewear device 502 via one or more connectors. The connectors can be wired or wireless connectors and can include electrical and/or non-electrical (e.g., structural) components. In some embodiments, eyewear device 502 and the wearable accessory device can operate independently without any wired or wireless connection between them.

In some situations, pairing external devices, such as an intermediary processing device (e.g., HIPD 206, 306, 406) with eyewear device 502 (e.g., as part of AR system 500) enables eyewear device 502 to achieve a similar form factor of a pair of glasses while still providing sufficient battery and computation power for expanded capabilities. Some, or all, of the battery power, computational resources, and/or additional features of AR system 800 can be provided by a paired device or shared between a paired device and eyewear device 502, thus reducing the weight, heat profile, and form factor of eyewear device 502 overall while allowing eyewear device 502 to retain its desired functionality. For example, the wearable accessory device can allow components that would otherwise be included on eyewear device 502 to be included in the wearable accessory device and/or intermediary processing device, thereby shifting a weight load from the user's head and neck to one or more other portions of the user's body. In some embodiments, the intermediary processing device has a larger surface area over which to diffuse and disperse heat to the ambient environment. Thus, the intermediary processing device can allow for greater battery and computation capacity than might otherwise have been possible on eyewear device 502 standing alone. Because weight carried in the wearable accessory device can be less invasive to a user than weight carried in the eyewear device 502, a user may tolerate wearing a lighter eyewear device and carrying or wearing the paired device for greater lengths of time than the user would tolerate wearing a heavier eyewear device standing alone, thereby enabling an artificial-reality environment to be incorporated more fully into a user's day-to-day activities.

AR systems can include various types of computer vision components and subsystems. For example, AR system 500 and/or VR system 600 can include one or more optical sensors such as two-dimensional (2D) or three-dimensional (3D) cameras, time-of-flight depth sensors, structured light transmitters and detectors, single-beam or sweeping laser rangefinders, 3D LiDAR sensors, and/or any other suitable type or form of optical sensor. An AR system can process data from one or more of these sensors to identify a location of a user and/or aspects of the use's real-world physical surroundings, including the locations of real-world objects within the real-world physical surroundings. In some embodiments, the methods described herein are used to map the real world, to provide a user with context about real-world surroundings, and/or to generate digital twins (e.g., interactable virtual objects), among a variety of other functions. For example, FIGS. 6A and 6B show VR system 600 having cameras 639A to 639D, which can be used to provide depth information for creating a voxel field and a two-dimensional mesh to provide object information to the user to avoid collisions.

In some embodiments, AR system 500 and/or VR system 600 can include haptic (tactile) feedback systems, which may be incorporated into headwear, gloves, body suits, handheld controllers, environmental devices (e.g., chairs or floormats), and/or any other type of device or system, such as the wearable devices discussed herein. The haptic feedback systems may provide various types of cutaneous feedback, including vibration, force, traction, shear, texture, and/or temperature. The haptic feedback systems may also provide various types of kinesthetic feedback, such as motion and compliance. The haptic feedback may be implemented using motors, piezoelectric actuators, fluidic systems, and/or a variety of other types of feedback mechanisms. The haptic feedback systems may be implemented independently of other artificial-reality devices, within other artificial-reality devices, and/or in conjunction with other artificial-reality devices.

In some embodiments of an artificial reality system, such as AR system 500 and/or VR system 600, ambient light (e.g., a live feed of the surrounding environment that a user would normally see) can be passed through a display element of a respective head-wearable device presenting aspects of the AR system. In some embodiments, ambient light can be passed through a portion less that is less than all of an AR environment presented within a user's field of view (e.g., a portion of the AR environment co-located with a physical object in the user's real-world environment that is within a designated boundary (e.g., a guardian boundary) configured to be used by the user while they are interacting with the AR environment). For example, a visual user interface element (e.g., a notification user interface element) can be presented at the head-wearable device, and an amount of ambient light (e.g., 15-50% of the ambient light) can be passed through the user interface element such that the user can distinguish at least a portion of the physical environment over which the user interface element is being displayed.

FIG. 8 is an illustration of an example system 800 that incorporates an eye-tracking subsystem capable of tracking a user's eye(s). As depicted in FIG. 8, system 800 may include a light source 802, an optical subsystem 804, an eye-tracking subsystem 806, and/or a control subsystem 808. In some examples, light source 802 may generate light for an image (e.g., to be presented to an eye 801 of the viewer). Light source 802 may represent any of a variety of suitable devices. For example, light source 802 can include a two-dimensional projector (e.g., a LCOS display), a scanning source (e.g., a scanning laser), or other device (e.g., an LCD, an LED display, an OLED display, an active-matrix OLED display (AMOLED), a transparent OLED display (TOLED), a waveguide, or some other display capable of generating light for presenting an image to the viewer). In some examples, the image may represent a virtual image, which may refer to an optical image formed from the apparent divergence of light rays from a point in space, as opposed to an image formed from the light ray's actual divergence.

In some embodiments, optical subsystem 804 may receive the light generated by light source 802 and generate, based on the received light, converging light 820 that includes the image. In some examples, optical subsystem 804 may include any number of lenses (e.g., Fresnel lenses, convex lenses, concave lenses), apertures, filters, mirrors, prisms, and/or other optical components, possibly in combination with actuators and/or other devices. In particular, the actuators and/or other devices may translate and/or rotate one or more of the optical components to alter one or more aspects of converging light 820. Further, various mechanical couplings may serve to maintain the relative spacing and/or the orientation of the optical components in any suitable combination.

In one embodiment, eye-tracking subsystem 806 may generate tracking information indicating a gaze angle of an eye 801 of the viewer. In this embodiment, control subsystem 808 may control aspects of optical subsystem 804 (e.g., the angle of incidence of converging light 820) based at least in part on this tracking information. Additionally, in some examples, control subsystem 808 may store and utilize historical tracking information (e.g., a history of the tracking information over a given duration, such as the previous second or fraction thereof) to anticipate the gaze angle of eye 801 (e.g., an angle between the visual axis and the anatomical axis of eye 801). In some embodiments, eye-tracking subsystem 806 may detect radiation emanating from some portion of eye 801 (e.g., the cornea, the iris, the pupil, or the like) to determine the current gaze angle of eye 801. In other examples, eye-tracking subsystem 806 may employ a wavefront sensor to track the current location of the pupil.

Any number of techniques can be used to track eye 801. Some techniques may involve illuminating eye 801 with infrared light and measuring reflections with at least one optical sensor that is tuned to be sensitive to the infrared light. Information about how the infrared light is reflected from eye 801 may be analyzed to determine the position(s), orientation(s), and/or motion(s) of one or more eye feature(s), such as the cornea, pupil, iris, and/or retinal blood vessels.

In some examples, the radiation captured by a sensor of eye-tracking subsystem 806 may be digitized (i.e., converted to an electronic signal). Further, the sensor may transmit a digital representation of this electronic signal to one or more processors (for example, processors associated with a device including eye-tracking subsystem 806). Eye-tracking subsystem 806 may include any of a variety of sensors in a variety of different configurations. For example, eye-tracking subsystem 806 may include an infrared detector that reacts to infrared radiation. The infrared detector may be a thermal detector, a photonic detector, and/or any other suitable type of detector. Thermal detectors may include detectors that react to thermal effects of the incident infrared radiation.

In some examples, one or more processors may process the digital representation generated by the sensor(s) of eye-tracking subsystem 806 to track the movement of eye 801. In another example, these processors may track the movements of eye 801 by executing algorithms represented by computer-executable instructions stored on non-transitory memory. In some examples, on-chip logic (e.g., an application-specific integrated circuit or ASIC) may be used to perform at least portions of such algorithms. As noted, eye-tracking subsystem 806 may be programmed to use an output of the sensor(s) to track movement of eye 801. In some embodiments, eye-tracking subsystem 806 may analyze the digital representation generated by the sensors to extract eye rotation information from changes in reflections. In one embodiment, eye-tracking subsystem 806 may use corneal reflections or glints (also known as Purkinje images) and/or the center of the eye's pupil 822 as features to track over time.

In some embodiments, eye-tracking subsystem 806 may use the center of the eye's pupil 822 and infrared or near-infrared, non-collimated light to create corneal reflections. In these embodiments, eye-tracking subsystem 806 may use the vector between the center of the eye's pupil 822 and the corneal reflections to compute the gaze direction of eye 801. In some embodiments, the disclosed systems may perform a calibration procedure for an individual (using, e.g., supervised or unsupervised techniques) before tracking the user's eyes. For example, the calibration procedure may include directing users to look at one or more points displayed on a display while the eye-tracking system records the values that correspond to each gaze position associated with each point.

In some embodiments, eye-tracking subsystem 806 may use two types of infrared and/or near-infrared (also known as active light) eye-tracking techniques: bright-pupil and dark-pupil eye tracking, which may be differentiated based on the location of an illumination source with respect to the optical elements used. If the illumination is coaxial with the optical path, then eye 801 may act as a retroreflector as the light reflects off the retina, thereby creating a bright pupil effect similar to a red-eye effect in photography. If the illumination source is offset from the optical path, then the eye's pupil 822 may appear dark because the retroreflection from the retina is directed away from the sensor. In some embodiments, bright-pupil tracking may create greater iris/pupil contrast, allowing more robust eye tracking with iris pigmentation, and may feature reduced interference (e.g., interference caused by eyelashes and other obscuring features). Bright-pupil tracking may also allow tracking in lighting conditions ranging from total darkness to a very bright environment.

In some embodiments, control subsystem 808 may control light source 802 and/or optical subsystem 804 to reduce optical aberrations (e.g., chromatic aberrations and/or monochromatic aberrations) of the image that may be caused by or influenced by eye 801. In some examples, as mentioned above, control subsystem 808 may use the tracking information from eye-tracking subsystem 806 to perform such control. For example, in controlling light source 802, control subsystem 808 may alter the light generated by light source 802 (e.g., by way of image rendering) to modify (e.g., pre-distort) the image so that the aberration of the image caused by eye 801 is reduced.

The disclosed systems may track both the position and relative size of the pupil (since, e.g., the pupil dilates and/or contracts). In some examples, the eye-tracking devices and components (e.g., sensors and/or sources) used for detecting and/or tracking the pupil may be different (or calibrated differently) for different types of eyes. For example, the frequency range of the sensors may be different (or separately calibrated) for eyes of different colors and/or different pupil types, sizes, and/or the like. As such, the various eye-tracking components (e.g., infrared sources and/or sensors) described herein may need to be calibrated for each individual user and/or eye.

The disclosed systems may track both eyes with and without ophthalmic correction, such as that provided by contact lenses worn by the user. In some embodiments, ophthalmic correction elements (e.g., adjustable lenses) may be directly incorporated into the artificial reality systems described herein. In some examples, the color of the user's eye may necessitate modification of a corresponding eye-tracking algorithm. For example, eye-tracking algorithms may need to be modified based at least in part on the differing color contrast between a brown eye and, for example, a blue eye.

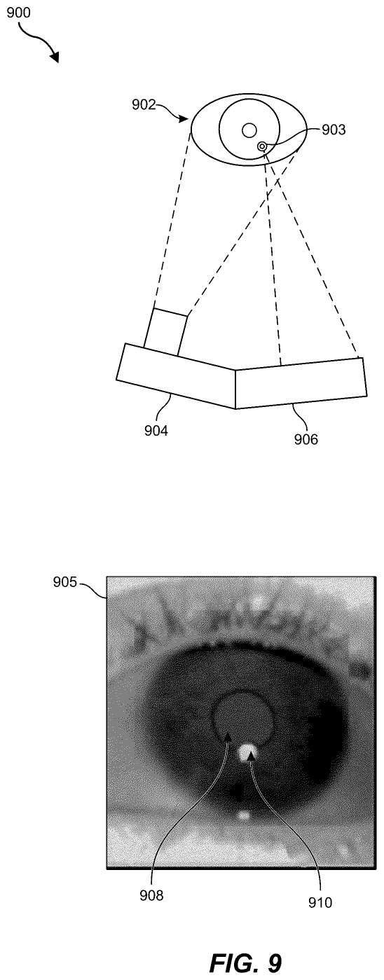

FIG. 9 is a more detailed illustration of various aspects of the eye-tracking subsystem illustrated in FIG. 8. As shown in this figure, an eye-tracking subsystem 900 may include at least one source 904 and at least one sensor 906. Source 904 generally represents any type or form of element capable of emitting radiation. In one example, source 904 may generate visible, infrared, and/or near-infrared radiation. In some examples, source 904 may radiate non-collimated infrared and/or near-infrared portions of the electromagnetic spectrum towards an eye 902 of a user. Source 904 may utilize a variety of sampling rates and speeds. For example, the disclosed systems may use sources with higher sampling rates in order to capture fixational eye movements of a user's eye 902 and/or to correctly measure saccade dynamics of the user's eye 902. As noted above, any type or form of eye-tracking technique may be used to track the user's eye 902, including optical-based eye-tracking techniques, ultrasound-based eye-tracking techniques, etc.

Sensor 906 generally represents any type or form of element capable of detecting radiation, such as radiation reflected off the user's eye 902. Examples of sensor 906 include, without limitation, a charge coupled device (CCD), a photodiode array, a complementary metal-oxide-semiconductor (CMOS) based sensor device, and/or the like. In one example, sensor 906 may represent a sensor having predetermined parameters, including, but not limited to, a dynamic resolution range, linearity, and/or other characteristic selected and/or designed specifically for eye tracking.

As detailed above, eye-tracking subsystem 900 may generate one or more glints. As detailed above, a glint 903 may represent reflections of radiation (e.g., infrared radiation from an infrared source, such as source 904) from the structure of the user's eye. In various embodiments, glint 903 and/or the user's pupil may be tracked using an eye-tracking algorithm executed by a processor (either within or external to an artificial reality device). For example, an artificial reality device may include a processor and/or a memory device in order to perform eye tracking locally and/or a transceiver to send and receive the data necessary to perform eye tracking on an external device (e.g., a mobile phone, cloud server, or other computing device).

FIG. 9 shows an example image 905 captured by an eye-tracking subsystem, such as eye-tracking subsystem 900. In this example, image 905 may include both the user's pupil 908 and a glint 910 near the same. In some examples, pupil 908 and/or glint 910 may be identified using an artificial-intelligence-based algorithm, such as a computer-vision-based algorithm. In one embodiment, image 905 may represent a single frame in a series of frames that may be analyzed continuously in order to track the eye 902 of the user. Further, pupil 908 and/or glint 910 may be tracked over a period of time to determine a user's gaze.

In one example, eye-tracking subsystem 900 may be configured to identify and measure the inter-pupillary distance (IPD) of a user. In some embodiments, eye-tracking subsystem 900 may measure and/or calculate the IPD of the user while the user is wearing the artificial reality system. In these embodiments, eye-tracking subsystem 900 may detect the positions of a user's eyes and may use this information to calculate the user's IPD.

As noted, the eye-tracking systems or subsystems disclosed herein may track a user's eye position and/or eye movement in a variety of ways. In one example, one or more light sources and/or optical sensors may capture an image of the user's eyes. The eye-tracking subsystem may then use the captured information to determine the user's inter-pupillary distance, interocular distance, and/or a 3D position of each eye (e.g., for distortion adjustment purposes), including a magnitude of torsion and rotation (i.e., roll, pitch, and yaw) and/or gaze directions for each eye. In one example, infrared light may be emitted by the eye-tracking subsystem and reflected from each eye. The reflected light may be received or detected by an optical sensor and analyzed to extract eye rotation data from changes in the infrared light reflected by each eye.

The eye-tracking subsystem may use any of a variety of different methods to track the eyes of a user. For example, a light source (e.g., infrared light-emitting diodes) may emit a dot pattern onto each eye of the user. The eye-tracking subsystem may then detect (e.g., via an optical sensor coupled to the artificial reality system) and analyze a reflection of the dot pattern from each eye of the user to identify a location of each pupil of the user. Accordingly, the eye-tracking subsystem may track up to six degrees of freedom of each eye (i.e., 3D position, roll, pitch, and yaw) and at least a subset of the tracked quantities may be combined from two eyes of a user to estimate a gaze point (i.e., a 3D location or position in a virtual scene where the user is looking) and/or an IPD.

In some cases, the distance between a user's pupil and a display may change as the user's eye moves to look in different directions. The varying distance between a pupil and a display as viewing direction changes may be referred to as “pupil swim” and may contribute to distortion perceived by the user as a result of light focusing in different locations as the distance between the pupil and the display changes. Accordingly, measuring distortion at different eye positions and pupil distances relative to displays and generating distortion corrections for different positions and distances may allow mitigation of distortion caused by pupil swim by tracking the 3D position of a user's eyes and applying a distortion correction corresponding to the 3D position of each of the user's eyes at a given point in time. Thus, knowing the 3D position of each of a user's eyes may allow for the mitigation of distortion caused by changes in the distance between the pupil of the eye and the display by applying a distortion correction for each 3D eye position. Furthermore, as noted above, knowing the position of each of the user's eyes may also enable the eye-tracking subsystem to make automated adjustments for a user's IPD.

In some embodiments, a display subsystem may include a variety of additional subsystems that may work in conjunction with the eye-tracking subsystems described herein. For example, a display subsystem may include a varifocal subsystem, a scene-rendering module, and/or a vergence-processing module. The varifocal subsystem may cause left and right display elements to vary the focal distance of the display device. In one embodiment, the varifocal subsystem may physically change the distance between a display and the optics through which it is viewed by moving the display, the optics, or both. Additionally, moving or translating two lenses relative to each other may also be used to change the focal distance of the display. Thus, the varifocal subsystem may include actuators or motors that move displays and/or optics to change the distance between them. This varifocal subsystem may be separate from or integrated into the display subsystem. The varifocal subsystem may also be integrated into or separate from its actuation subsystem and/or the eye-tracking subsystems described herein.

In one example, the display subsystem may include a vergence-processing module configured to determine a vergence depth of a user's gaze based on a gaze point and/or an estimated intersection of the gaze lines determined by the eye-tracking subsystem. Vergence may refer to the simultaneous movement or rotation of both eyes in opposite directions to maintain single binocular vision, which may be naturally and automatically performed by the human eye. Thus, a location where a user's eyes are verged is where the user is looking and is also typically the location where the user's eyes are focused. For example, the vergence-processing module may triangulate gaze lines to estimate a distance or depth from the user associated with intersection of the gaze lines. The depth associated with intersection of the gaze lines may then be used as an approximation for the accommodation distance, which may identify a distance from the user where the user's eyes are directed. Thus, the vergence distance may allow for the determination of a location where the user's eyes should be focused and a depth from the user's eyes at which the eyes are focused, thereby providing information (such as an object or plane of focus) for rendering adjustments to the virtual scene.

The vergence-processing module may coordinate with the eye-tracking subsystems described herein to make adjustments to the display subsystem to account for a user's vergence depth. When the user is focused on something at a distance, the user's pupils may be slightly farther apart than when the user is focused on something close. The eye-tracking subsystem may obtain information about the user's vergence or focus depth and may adjust the display subsystem to be closer together when the user's eyes focus or verge on something close and to be farther apart when the user's eyes focus or verge on something at a distance.

The eye-tracking information generated by the above-described eye-tracking subsystems may also be used, for example, to modify various aspect of how different computer-generated images are presented. For example, a display subsystem may be configured to modify, based on information generated by an eye-tracking subsystem, at least one aspect of how the computer-generated images are presented. For instance, the computer-generated images may be modified based on the user's eye movement, such that if a user is looking up, the computer-generated images may be moved upward on the screen. Similarly, if the user is looking to the side or down, the computer-generated images may be moved to the side or downward on the screen. If the user's eyes are closed, the computer-generated images may be paused or removed from the display and resumed once the user's eyes are back open.

The above-described eye-tracking subsystems can be incorporated into one or more of the various artificial reality systems described herein in a variety of ways. For example, one or more of the various components of system 800 and/or eye-tracking subsystem 900 may be incorporated into any of the augmented-reality systems in and/or virtual-reality systems described herein in to enable these systems to perform various eye-tracking tasks (including one or more of the eye-tracking operations described herein).

As stated, HMDs are ubiquitous and used in a wide variety of technological fields, e.g., gaming and entertainment, manufacturing, education, military and defense, aviation, and healthcare. In healthcare, one or more components of HMDs, operating independently or in conjunction with other devices, can be utilized to determine various physiological parameters of an individual such as blood pressure, heart rate, pulse rate, and so forth. Various challenges, however, complicate the ability of these devices to obtain, analyze, and accurately determine these parameters. For example, a whole slew of components need to be installed on and integrated with the existing operation of the HMDs, which imposes various constraints on these HMDs, namely increased size and weight. Consequently, user comfort is reduced and user experience is adversely affected. Moreover, additional new hardware involves installation of more complex software applications and architectures and need to be integrated with the existing software infrastructure operating in these HMDs, resulting in higher power consumption levels.

The inclusion of additional hardware devices also increases systemic inefficiencies because the software architecture of the HMDs may need to manage multiple component-specific applications, prioritize competing tasks, and sometimes interrupt or halt ongoing processes to accommodate new sensor inputs. These operations can overburden the device's memory and processing resources, leading to slower performance, reduced battery life, and a diminished user experience. As a result, techniques that enable accurate and approximately real-time determination of physiological parameters—e.g., such as heart rate—using current hardware of the HMDs, while maintaining device comfort and system efficiency, is desirable.

The techniques described herein address and overcome the above listed deficiencies by enabling HMDs to determine various physiological parameters, e.g., a heart rate, using the existing hardware infrastructures of the HMDs. Specifically, these techniques facilitate non-contact and approximately real-time measurement of physiological parameters by utilizing inward-facing cameras of the HMDs that are used for eye or face tracking applications, thereby eliminating the need for additional contact-based sensors. This preserves device comfort, reduces hardware complexity, and minimizes power consumption. The system intelligently selects, tracks, and modifies a region of interest on a user's face, adapting to movement and anatomical differences, while implementing advanced signal processing techniques to filter noise from ambient light and motion artifacts to ensure reliable signal quality.

Additionally, the techniques described herein provide a specific and practical technological improvement to the functioning of head-mounted devices. By integrating various signal processing algorithms with existing hardware in a manner that enables approximately real-time, non-contact based physiological parameter determination, these techniques provide concrete technical challenges-such as dynamic region of interest selection and modification, compensation for user movement, robust noise filtering, accurate gathering of the underlying data utilized to determine various physiological parameters, and the implementation of a combination of various signal processing algorithms to determine the physiological parameter values.

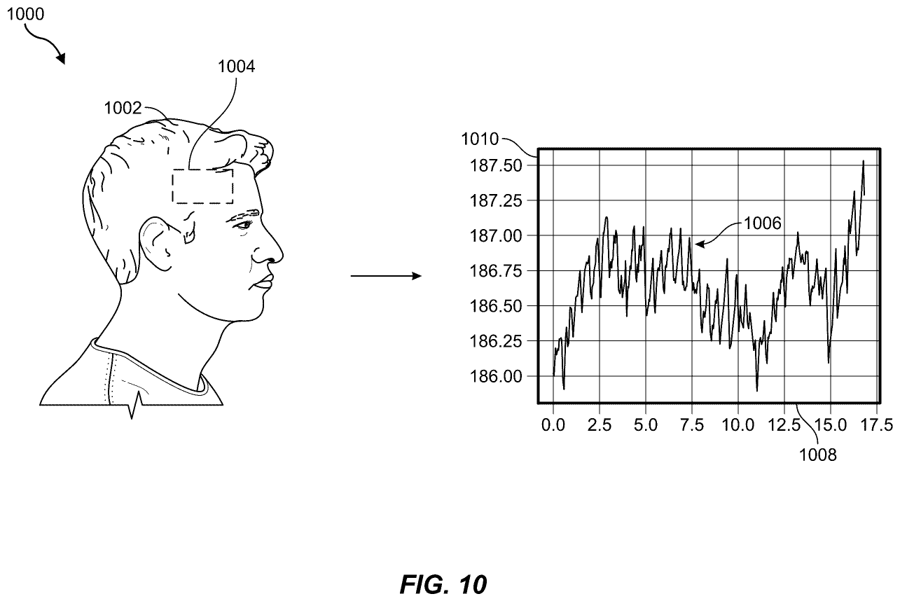

FIG. 10 depicts an example implementation of physiological parameter determination system 1000 (hereinafter “system 1000”), according to some aspects described and illustrated herein. In aspects, a user wearing an HMD (e.g., included as part of AR System 500 or VR system 600) may interact with content included as part of a virtual reality or augmented reality environment. As part of or independent of this interaction, one or more of the cameras (one or more cameras 738A or cameras 738A) are utilized by the HMD to track eye movement of the user. For example, the eye tracking feature is utilized to assist user 1002 navigate a virtual (or real) environment, select objects within this environment, and control various interactive icons displayed at various locations in this environment.

Implementing the eye tracking feature involves continuously or periodically obtaining images (or a live video stream), approximately in real time (within a second or fractions of a second), of the facial region in order to analyze the position and movement of the eyes of user 1002. This data is utilized to accurately estimate a direction of his gaze in order to determine a point of the user's focus within the virtual reality or augmented reality environment. Physiological parameter determination system 1000 determines, using processor 748A, light intensity data specific to a region on the face of user 1002 (e.g., region of interest) by analyzing one or more of the images or live video stream. Thereafter, physiological parameter determination system 1000 utilizes this data to determine or more physiological parameters specific to user 1002.

In operation, image data representative of the images obtained by cameras 738A are routed to memory 750A and accessed by processor 748A for further processing. For example, processor 748A executes one or more software applications or instructions, automatically and without user intervention, on the image data to identify a region of interest on the user's facial region. Various factors are examined by processor 748A in identifying the region of interest, e.g., the ease with which changes in blood volume can be determined in the region (affected to a large extent by the density of blood vessels), lighting conditions, and so forth. Moreover, processor 748A can dynamically change, approximately in real time, the selected region of interest based on a threshold level of variation in any of the above factors.

For example, if a particular user has a tendency to frequently alter his or her facial expression near his or her forehead, processor 748A can select another facial area as the region of interest, e.g., cheeks, temples, etc. Similarly, if lighting conditions are unfavorable for area of the facial region (e.g., temples) or the anatomical characteristics of a particular user indicate that the area above the forehead has a higher concentration of blood vessels than the temples, processor 748A selects the area above the forehead as the region of interest. It is noted that processor 748A may consider other factors as well. In operation, processor 748A may compare light intensity values associated with images of multiple regions of the face of user 1002, e.g., cheek and forehead, and determine that the light intensity values reflected from the forehead is stronger than the light intensity values reflected from the cheek, and as such, may discard the light intensity values associated with the cheek and store the light intensity values reflected from the forehead. In aspects, processor 748A may compare each set of light intensity values relative to a threshold value in addition to comparing the light values reflected from the forehead and cheek of the user. For example, if processor 748A determines the light intensity values as being below a threshold value, processor 748A may discard these entries, e.g., by deleting these from memory 750A.

Returning to FIG. 10, processor 748A, after analyzing one or more of the above factors, identifies forehead area 1004 as the region of interest. Having selected the region of interest as the forehead area 1004, processor 748A extracts light intensity values specific to this area over a particular time frame for further processing. In operation, processor 748A, after selecting the region of interest as forehead area 1004, accesses image data of each of a plurality of images (or a live video stream) captured by cameras 738A and identifies a light intensity value specific to each pixel of a number of pixels that are included in forehead area 1004. Stated differently, processor 748A extracts and identifies light intensity values for pixels that are included as part of the forehead area 1004 from image data representative of a number of different images of user 1002. The processor 748A then generates light intensity graphical representation 1006 by averaging these intensity values for each of the plurality of images. For each image, processor 748A determines an average of the light intensity values of the pixels that define the region of interest-forehead area 1004. The light intensity graphical representation 1006 includes x-axis 1008 and y-axis 1010. In aspects, the x-axis lists time values ranging from 0.0 to 17.5 seconds and the y-axis lists light intensity values ranging from 186.00 to 187.50.

It is noted that these light intensity values represent grayscale values that range from 0 to 255. For example, for monochrome cameras, a light intensity of each pixel is represented by a distinct grayscale value such that a value of 0 represent no light, while a value of 255 represent a maximum amount of light intensity. In contrast, for color cameras, each pixel can be represented by three color channels—red, green, and blue—and each value can ranges from 0 to 255. Processor 748A may output light intensity graphical representation 1006 via display 735A. Thereafter, each average light intensity value is included as part of light intensity graphical representation 1006. These light intensity values are numerical representations of amounts of light reflected back from a surface (e.g., forehead area 1004) and captured by one or more sensors of cameras 738A. As part of implementing the process or capturing the plurality of images (or the live video stream), cameras 738A emit light directly onto areas of which cameras 738A obtain images, and detect light that is reflected back. In operation, some of the light that is emitted is absorbed by these areas while the rest of the light is reflected from the surface. At least some of the reflected light is detected by one or more sensors of cameras 738A and included in image data routed to memory 750A. Processor 748A then extracts and processes this data.

Specifically, Processor 748A, upon completion of the extraction of light intensity values specific to each pixel included in forehead area 1004, determines that a subset of these light intensity values includes data relevant for determining one or more physiological parameters (e.g., heartbeat), while identifying at least some of these values as being unrelated to physiological parameters. For example, a subset of these light intensity values correspond to movement artifacts, respiration, ambient light fluctuations, a drift or baseline shift in a position of one or more sensors, and so forth. Movement artifacts can result from a sudden shift in the position of the face of user 1002, while ambient light fluctuations can result from gradual or sudden changes in various environment conditions, e.g., sunlight, room lights, shadows, and so forth. Such ambient light fluctuations create noticeable variations in light intensity values. Processor 748A, in order to compensate for these variations, and by extension, more accurately determine one or more physiological parameters of user 1002, implements one or more additional processing or filtering operations.

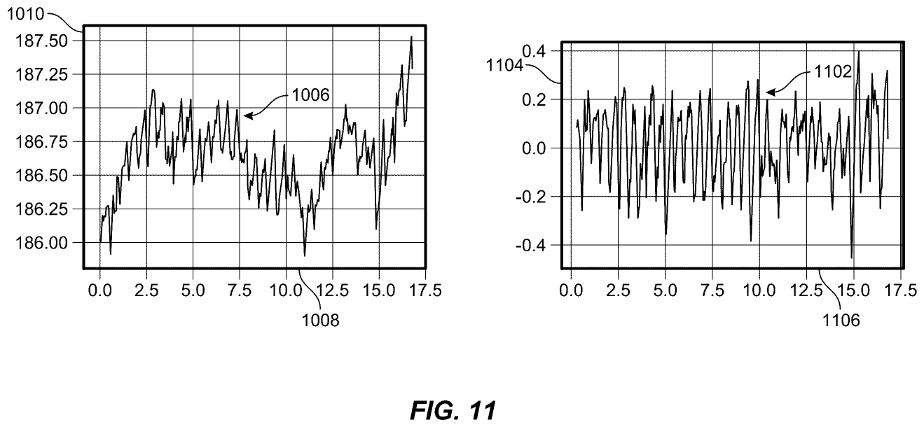

FIG. 11 illustrates filtered graphical representation 1102, according to some aspects described and illustrated herein. In aspects, processor 748A, operating independently or in conjunction with one or more external devices, implements a moving or rolling average operation on data included as part of light intensity graphical representation 1006. For example, if cameras 739A captured 40 images frames (image), processor 748A may determine 40 different light intensity values from these 40 images and include these values as part of light intensity graphical representation 1006. In operation, processor 748A may generate filtered graphical representation 1102 via a multiple step process. First, processor 748A may identify a moving average window, e.g., 5 image frames. Second, processor 748A may determine an average value of the first five light intensity values of image frames 1, 2, 3, 4, and 5 and then subtract the averaged value from the light intensive value specific to image 5 as included in light intensity graphical representation 1006. For example, if the light intensity values of images 1, 2, 3, 4, and 5 are 185.5, 185.7, 185.9, 186.1 and 186.2, processor 748A determines an average of these values—185.88 in this instance—and then subtracts the determined average value (185.88) from the light intensity value of 186.2 as included in light intensity graphical representation 1006 to determine a deviation value.

Third, processor 748A iteratively performs this moving average operation on images frames 6-40 using the predefined image frame window of 5. Specifically, for image frame 6, processor 748A averages light intensity values of image frames 2-6 and subtracts the determined average value from the light intensity value of image frame 6 (as included in light intensity graphical representation 1006) to determine another deviation value. Similarly, for image frame 7, processor 748A averages light intensity values of image frames 3-7 and subtracts the determined average value from the light intensity value of image frame 7 (as included in light intensity graphical representation 1006) to determine yet another deviation value. In this way, processor 748A determines deviation values for all 40 image frames and includes them as part of filtered graphical representation 1102. Fourth, processor 748A includes each deviation value associated with each of the 40 image frames on filtered graphical representation 1102 such that these deviations values are associated with y-axis 1104 and correspond to the time values (associated with the predefined image frame window of 5). The times values are plotted along x-axis 1106. Processor 748A may output filtered graphical representation 1102 via display 735A.

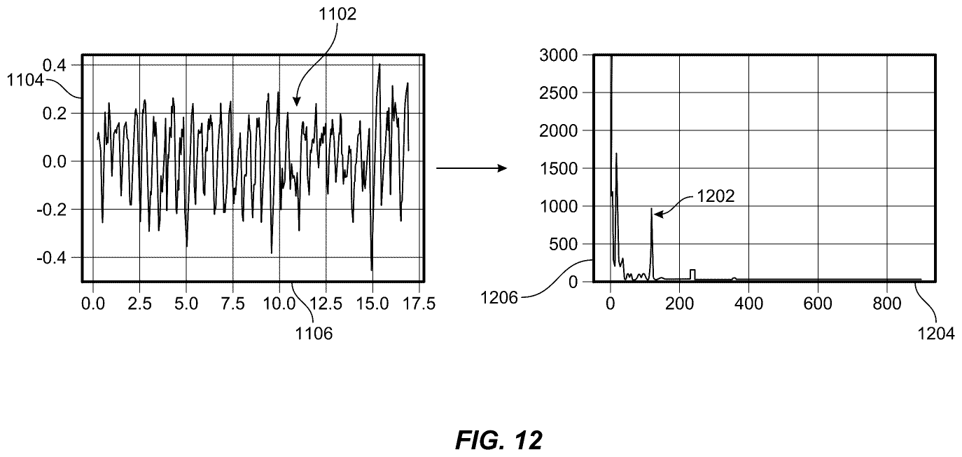

FIG. 12 depicts a frequency graphical representation derived from filtered graphical representation 1102, according to some aspects described and illustrated herein. In aspects, processor 748A implements a Fourier transform algorithm on the values included as part of filtered graphical representation 1102 to generate frequency value graphical representation 1202. Specifically, processor 748A, by implementing a Fast Fourier Transform (FFT) algorithm on the times series of values included as part of filtered graphical representation 1102, namely the deviation values (light intensity deviation values) of the captured image frames determined over a particular time frame, transforms the values of filtered graphical representation 1102 from a time domain to a frequency domain.

In operation, using the time values of filtered graphical representation 1102 as associated with x-axis 1106 and deviation values associated with y-axis 1104 of representation 1102 as inputs to the FFT transform algorithm, processor 748A generates outputs in the form of frequency values (in Hertz or Hz) and amplitude values (which share the same unit as deviation values—grayscale or color values ranging from 0 to 255 bytes). Processor 748A includes these frequency and amplitude values—values that correspond to the light intensity deviations and time values included in filtered graphical representation 1102—as part of frequency graphical representation 1202 such that the frequency values are associated with x-axis 1204 and the amplitude values are associated with y-axis 1206. Thereafter, processor 748A determines, automatically and without user intervention, an amplitude having a highest value as corresponding to a physiological parameter specific to user 1002, e.g., heartbeat.

In particular, processor 748A determines the highest value as corresponding to heartbeat of user 1002 because this value corresponds to a dominant and repetitive signal derived from the light intensity deviation data. Additionally, processor 748A, by implementing the filtering operation described above and illustrated in FIG. 11, removes signals that may be repetitive but which are associated with movement artifacts, respiration, ambient light fluctuations, and so forth. In this way, processor 748A determines, subsequent to completion of the filtering operation, that the dominant and repetitive signal derived from the light intensity deviation data of filtered graphical representation 1102 most likely corresponds to the heartbeat of user 1002.

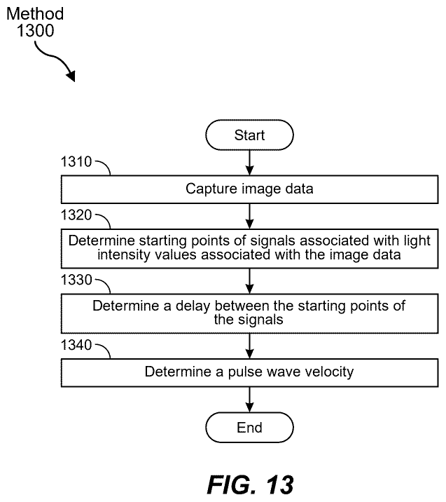

FIG. 13 depicts a method 1300 for determining another physiological parameter of a user, according to some aspects described and illustrated herein. In additional to cameras 738A, various sensors (not shown) may be positioned on different locations on the body of user 1002. For example, sensors capable of capturing image data of subtle changes in skin color or light reflecting from parts of a user's body may be suitable for implemented the method 1300. In aspects, in addition to cameras 738A, a camera may be positioned adjacent an earlobe of user 1002 and another camera may be positioned on or adjacent a finger of user 1002. Each of these cameras may be communicatively coupled to processor 748A and integrated as part of system 720 and physiological parameter determination system 1000. The integration may include hardware integration and/or software integration such that processor 748A may access data stored in local memory of each of the camera positioned on the earlobe of user 1002 and the camera positioned on the finger of user 1002 via wired or wireless connection.

In operation, at step 1310, processor 748A controls each of the earlobe location camera and the finger location camera such that processor 748A, automatically and without user intervention, instructs both cameras to begin capturing image data of areas associated with the earlobe and the finger of user 1002. In aspects, each camera captures an image, a sequence of images for a predetermined time frame, a live video stream for the predetermined time frame, and so forth, of the earlobe and finger areas, respectively. Processor 748A determines average light intensity values of pixels defining portions of various regions of interest of the earlobe and the finger of user 1002 by accessing the image data of the images captured by the earlobe location camera and the finger location camera. The manner in which the average light intensity values are determined are similar to the techniques described above and illustrated at least in FIG. 11.

At step 1320, processor 748A determines starting points of the signals associated with the light intensity values of the areas associated with the earlobe (starting point 1) and those associated with the finger (starting point 2). In aspects, each starting point corresponds to a portion of each signal from which there is a sudden and sharp increase, indicating an arrival of, e.g., a pulse wave. In aspects, processor 748A may implement one or more of a plurality of signal processing techniques to determine this portion, e.g., derivative analysis, second derivative analysis, thresholding, wavelet transform, matched filtering, ensemble averaging, or zero-crossing detection.

At step 1330, processor 748A determines a difference in time (e.g., a delay) between the starting point 1 and starting point 2 and designates this delay as a pulse transit time (PTT). PTT corresponds to the time that it takes a signal to travel from one location on the body of user 1002 to the other. In this instance, PTT represents the time that it would take for the signal associated with the light intensity values of the areas associated with the earlobe to travel to the finger and that of the signal associated with the light intensity values of the areas associated with the finger to travel to the earlobe.

Finally, at step 1340, processor 748A determines a pulse wave velocity by dividing a physical distance between the location of the camera for capturing areas associated with the earlobe and that of the camera for capturing areas associated with the finger, by the PTT. In aspects, the distance is stored locally in memory of each of the cameras, and in memory 750A. Processor 748A utilizes the pulse wave velocity to determine a blood pressure value of user 1002. There is a correlation between the pulse wave velocity and blood pressure in that a higher pulse wave velocity, compared to a baseline threshold, results in a higher blood pressure and a lower pulse wave velocity relative to the baseline threshold result in a lower blood pressure value. In short, the pulse wave velocity is proportional to blood pressure behavior.

In some embodiments, the various methods and systems described herein may be performed wholly or in part by a hardware processor executing software instructions stored in a memory. Such operations may be performed within a server or other cloud-accessible device, a desktop or laptop computer, a tablet computer, a smartphone, etc.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to any claims appended hereto and their equivalents in determining the scope of the present disclosure.

Unless otherwise noted, the terms “connected to” and “coupled to” (and their derivatives), as used in the specification and/or claims, are to be construed as permitting both direct and indirect (i.e., via other elements or components) connection. In addition, the terms “a” or “an,” as used in the specification and/or claims, are to be construed as meaning “at least one of.” Finally, for ease of use, the terms “including” and “having” (and their derivatives), as used in the specification and/or claims, are interchangeable with and have the same meaning as the word “comprising.”

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A head-mounted device comprising:
a sensor that detects light emanating from a region of interest of a face of a user during a time frame; and
a processor configured to:
determine, based on the light that is detected, a sequence of light intensity values over the time frame, and
determines a heart rate of a user using the light intensity values.

2. The head-mounted device of claim 1, further comprising:
a camera; and
the sensor that detects light being included as part of the camera.

3. The head-mounted device of claim 1, wherein the processor determines the heart rate of the user using the light intensity values at least in part by determining average values of the light intensity values.

4. The head-mounted device of claim 3, wherein the determining of the heart rate by the processor further comprises implementing, by the processor, a Fourier transform on the average values.

5. The head-mounted device of claim 4, wherein the determining of the heart rate by the processor further comprises:
the processor:
generating, based on the implementing of the Fourier transform on the average values, a plurality of frequencies and a plurality of amplitudes.

6. The head-mounted device of claim 5, wherein the processor is further configured to:
identify, as the heart rate of the user, an amplitude from the plurality of amplitudes that has a magnitude that is higher than respective magnitudes of a remaining amplitudes of the plurality of amplitudes.

7. The head-mounted device of claim 1, wherein the region of interest is associated with a forehead of the user.

8. The head-mounted device of claim 1, wherein the region of interest corresponds to at least one of the cheeks, temples, or periocular region of the user.

9. A head-mounted device comprising:
a sensor that detects light emanating from a first region on a face of a user during a time frame;
an additional sensor that detects additional light emanating from a second region of interest on the face of the user during the time frame; and
a processor, wherein the processor:
determines light intensity values from the light that reflected from the first region,
determines additional light intensity values from the additional light reflected from the additional region, and
determines a heart rate of a user using the light intensity values or the additional light intensity values.

10. The head-mounted device of claim 9, wherein the determining of the heart rate by the processor comprises the processor comparing the light intensity values associated with the first region to the additional light intensity values associated with the additional region.

11. The head-mounted device of claim 10, wherein the determining of the heart rate by the processor further comprises the processor:
discarding the additional light intensity values responsive to the additional light intensity values being lower than the light intensity values.

12. The head-mounted device of claim 11, wherein the determining of the heart rate by the processor further comprises the processor:
determining average values of the light intensity values; and
implementing a Fourier transform on the average values.

13. The head-mounted device of claim 12, wherein the determining of the heart rate by the processor further comprises the processor:
generating, based on the implementing of the Fourier transform on the average values, a plurality of frequencies and a plurality of amplitudes; and
identifying as the heart rate of the user, an amplitude from the plurality of amplitudes that has a magnitude that is higher than respective magnitudes of a remaining amplitudes of the plurality of amplitudes.

14. The head-mounted device of claim 9, wherein the first region and the second region are located on the face of the user.

15. The head-mounted device of claim 9, wherein the first region corresponds to at least one of the cheeks, temples, or periocular region of the user.

16. A method implementing by a computing device, the method comprising:
detecting, by a camera communicatively coupled with the computing device, light reflecting from a region of interest of a face of a user during a time frame;
determining by the computing device, based on the light that is detected, a sequence of light intensity values over the time frame, and
determining, by the computing device, a heart rate of a user using the light intensity values.

17. The method of claim 16, wherein the determining of the heart rate comprises:
determining average values of the light intensity values; and
implementing a Fourier transform on the average values.

18. The method of claim 17, wherein the determining of the heart rate further comprises:
generating by the computing device, based on the implementing of the Fourier transform on the average values, a plurality of frequencies and a plurality of amplitudes.

19. The method of claim 18, further comprising:
identifying as the heart rate of the user, by the computing device, an amplitude from the plurality of amplitudes that has a magnitude that is higher than respective magnitudes of a remaining amplitudes of the plurality of amplitudes.

20. The method of claim 16, further comprising:
detecting, by an inertial measurement unit communicatively coupled to the computing device, inertial data specific to the user within a particular time frame;
processing, by the computing device, the inertial data specific to the user and a plurality of images captured by the camera, the processing including implementing an artificial intelligence based machine learning model on the inertial data and the plurality of images; and
generating responsive to the processing, by the computing device, at least a blood pressure value specific to the user.
